(19) United States
(12) Patent Application Publication
(10) Pub. No.: US 2023/0215565 A1
(43) Pub. Date: Jul. 6, 2023

(54) HEALTH STATE ESTIMATION USING MACHINE LEARNING

(71) Applicant: ROCKLEY PHOTONICS LIMITED, Altrincham (GB)

(72) Inventors: Casimir WIERZYNSKI, La Jolla, CA (US); Daniel Carl GRADY, San Diego, CA (US); Sangshik PARK, Irvine, CA (US)

(21) Appl. No.: 17/823,505

(22) Filed: Aug. 30, 2022

Related U.S. Application Data
(60) Provisional application No. 63/239,657, filed on Sep. 1, 2021.

(51) Int. Cl. G16H 50/20; G06N 3/088; G06N 3/044; G16H 40/63

(52) U.S. Cl. CPC G16H 50/20; G06N 3/044; G06N 3/088; G16H 40/63

(57) ABSTRACT

A system and method for health state estimation. In some embodiments, the method includes receiving a first measurement of a subject, the first measurement being a first tissue spectrum of the subject; and generating, using a machine learning inference process based on the first measurement, an estimate of an aspect of the health state of the subject.

### CROSS-REFERENCE TO RELATED APPLICATION(S)

The present application claims priority to and the benefit of U.S. Provisional Application No. 63/239,857, filed Sep. 1, 2021, entitled “HEALTH STATE ESTIMATION USING MACHINE LEARNING”, the entire content of which is incorporated herein by reference.

### FIELD

One or more aspects of embodiments according to the present disclosure relate to health monitoring, and more particularly to a system and method for health state estimation using machine learning.

### BACKGROUND

Related art methods for monitoring indicators of patient health, e.g., by healthcare providers, may have various drawbacks. For example, a healthcare provider may receive new information regarding the health of a patient only relatively rarely, and, in some instances, as a result of an adverse change in the health of a patient, resulting in perceptible symptoms. Diagnosing and treating a health condition only after the patient experiences symptoms may result in potentially needless patient suffering, and, in some cases, increased treatment challenges.

Thus, there is a need for an improved system and method for monitoring indicators of patient health.

### SUMMARY

According to an embodiment of the present disclosure, there is provided a method, including: receiving a first measurement of a subject, the first measurement being a first tissue spectrum of the subject; and generating, using a machine learning inference process based on the first measurement, an estimate of an aspect of the health state of the subject.

In some embodiments, the aspect of the health state of the subject is a concentration of a chemical constituent of a tissue of the subject.

In some embodiments, the chemical constituent is a substance selected from the group consisting of glucose, cortisol, cholesterol, lactate, ethanol, and water.

In some embodiments, the aspect of the health state of the subject is the presence of a medical condition.

In some embodiments, the machine learning inference process is an inference process of a machine learning model selected from the group consisting of neural networks, support vector machines, partial least squares algorithms, decision trees, and combinations thereof.

In some embodiments, the method further includes receiving a second measurement of the subject, wherein the machine learning inference process is further based on the second measurement.

In some embodiments, the second measurement is a second tissue spectrum of the subject.

In some embodiments, the first tissue spectrum and the second tissue spectrum are obtained at different points in time.

In some embodiments, the first tissue spectrum and the second tissue spectrum are obtained at different locations on the body of the subject.

In some embodiments, the second measurement of the subject is a result of a chemical analysis of a sample from the subject.

In some embodiments, the second measurement of the subject is an image of a portion of the subject.

In some embodiments, the method further includes performing a machine learning training process to generate a trained state, wherein the machine learning training process is based on a plurality of measurements of one or more training subjects; and the machine learning inference process is further based on the trained state.

In some embodiments, the machine learning training process is further based on a plurality of target variables, each of the target variables corresponding to a respective measurement of the plurality of measurements.

In some embodiments, the machine learning training process is further based on a measurement of the subject, and a target variable corresponding to the measurement of the subject.

In some embodiments, the machine learning training process comprises clustering.

In some embodiments, the machine learning training process comprises dimensionality reduction.

According to an embodiment of the present disclosure, there is provided a method, including: receiving a first measurement of a subject; receiving a second measurement of the subject, obtained after the first measurement; and generating, using a machine learning inference process based on the first measurement and on the second measurement, an estimate of an aspect of the health state of the subject.

In some embodiments, the aspect of the health state of the subject is a concentration of a chemical constituent of a tissue of the subject.

In some embodiments, the chemical constituent is a substance selected from the group consisting of glucose, cortisol, cholesterol, lactate, ethanol, and water.

In some embodiments, the aspect of the health state of the subject is the presence of a medical condition.

In some embodiments: the first measurement is a first tissue spectrum of the subject, and the second measurement is a second tissue spectrum of the subject.

In some embodiments, the method further includes receiving a third measurement of the subject, wherein the machine learning inference process is further based on the third measurement.

In some embodiments: the third measurement is a third tissue spectrum of the subject, and the first tissue spectrum and the third tissue spectrum are obtained at different locations on the body of the subject.

In some embodiments: the first measurement of the subject is a result of a chemical analysis of a first sample from the subject, and the second measurement of the subject is a result of a chemical analysis of a second sample from the subject.

In some embodiments: the first measurement of the subject is a first image of a portion of the subject, and the second measurement of the subject is a second image of a portion of the subject.

In some embodiments, the method further includes performing a machine learning training process to generate a trained state, wherein the machine learning training process is based on a plurality of measurements of one or more training subjects; and the machine learning inference process is further based on the trained state.

In some embodiments, the machine learning training process is further based on a plurality of target variables, each of the target variables corresponding to a respective measurement of the plurality of measurements.

In some embodiments, the machine learning training process comprises clustering.

In some embodiments, the machine learning training process comprises dimensionality reduction.

In some embodiments, the machine learning inference process is an inference process of a machine learning model selected from the group consisting of neural networks, support vector machines, partial least squares algorithms, decision trees, and combinations thereof.

In some embodiments, the machine learning inference process is an inference process of a recurrent neural network.

According to an embodiment of the present disclosure, there is provided a system, including: a processing circuit, the processing circuit being configured to: receive a first measurement of a subject, the first measurement being a first tissue spectrum of the subject; and generate, using a machine learning inference process based on the first measurement, an estimate of an aspect of the health state of the subject.

In some embodiments, the aspect of the health state of the subject is a concentration of a chemical constituent of a tissue of the subject.

In some embodiments, the aspect of the health state of the subject is the presence of a medical condition.

In some embodiments: the processing circuit is further configured to receive a second measurement of a subject, the second measurement being a second tissue spectrum of the subject, obtained later than the first measurement; and the generating of the estimate of the aspect of the health state of the subject includes using a machine learning inference process further based on the second measurement.

In some embodiments, the system further includes a portable spectrophotometer, configured to obtain a plurality of tissue spectra including the first tissue spectrum.

### BRIEF DESCRIPTION OF THE DRAWINGS

These and other features and advantages of the present disclosure will be appreciated and understood with reference to the specification, claims, and appended drawings wherein:

FIG. 1 is a flow chart of a method for training, according to an embodiment of the present disclosure;

FIG. 2 is a flow chart of a method for inference, according to an embodiment of the present disclosure; and

FIG. 3 is a block diagram of a system including a central server and a plurality of sensors, according to an embodiment of the present disclosure.

### DETAILED DESCRIPTION

The detailed description set forth below in connection with the appended drawings is intended as a description of exemplary embodiments of a system and method for monitoring indicators of patient health provided in accordance with the present disclosure and is not intended to represent the only forms in which the present disclosure may be constructed or utilized. The description sets forth the features of the present disclosure in connection with the illustrated embodiments. It is to be understood, however, that the same or equivalent functions and structures may be accomplished by different embodiments that are also intended to be encompassed within the scope of the disclosure. As denoted elsewhere herein, like element numbers are intended to indicate like elements or features.

In related art medical care settings, a physician may observe a patient to make diagnoses and prescribe treatments. These observations can be costly in terms of time and resources, and they may be invasive, leading to patient discomfort and potentially causing the patient to be reluctant to participate. As a result, such observations may be carried out infrequently, e.g., once per year, especially if the patient has no reason to believe that they are sick. In part because of this paucity of observations, disease states may present themselves once patient illnesses are advanced and more difficult to treat.

Some embodiments address this problem by combining a biometric sensor (e.g., a wearable, non-invasive sensor) with a suitable analysis platform (e.g., a cloud-based analysis platform). The analysis platform may be a suitable machine learning model, which may be trained to infer a health outcome (e.g., the presence of a medical condition, such as diabetes) from measurements of a subject (e.g., a patient). The training may involve receiving measurements, along with reports of one or more health outcomes, from a plurality of subjects (or “training subjects”), and training the machine learning model to infer, from other measurements, the presence (or absence) of the health outcome. For example, during training, the machine learning model may receive measurements of each of a plurality of training subjects who may also report whether or not they have diabetes. The measurements may be generated by a wearable spectrophotometer; such measurements may be referred to as “tissue spectra” of the subject or subjects, as discussed in further detail below. The tissue spectra may be obtained by reflectance from or transmission though the tissue. Such spectra may be absorption spectra and may be diffuse reflectance absorption spectra. Furthermore, the tissue spectra may be one or more of a variety of spectral types including fluorescence spectra and Raman spectra. Once the machine learning model has been trained, it may be able to infer, from a tissue spectrum of another subject, various aspects of the health state of a subject, e.g., whether the subject has diabetes (or the severity of the diabetes, if present).

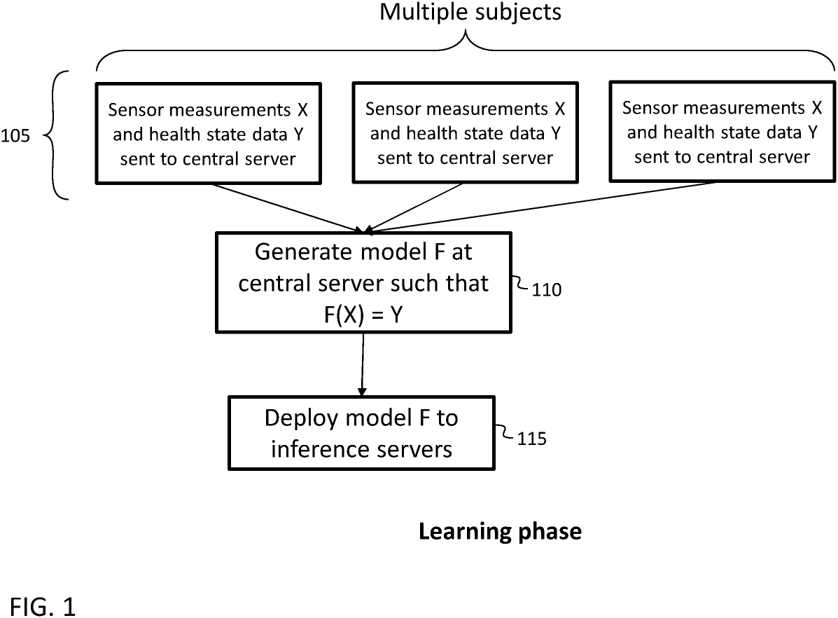

A measurement from a wearable spectrophotometer (or other measurements, as discussed in further detail below) may be used as an input observation. Training data including a set of such input observations, and a corresponding set of target variables, may be used to train a machine learning model, such as a neural network. Referring to FIG. 1, this training, or “learning” phase may be supervised training in which the training data set 105 includes, for each of a plurality of training subjects (e.g., patients), an input observation, which may be a set of measurements (e.g., one or more tissue spectra) X, and a set of target variables, which may be data Y regarding one or more aspects of a subject’s medical state. As used herein, a “target variable” is one or more numbers, used to train a machine learning model, each representing an aspect of the health state (the aspect being an aspect to be estimated, during inference, by the machine learning model) of the subject. An “estimate” is the output of a machine learning model when the machine learning model receives an input observation. An estimate may be a single number or a set of numbers. As used herein, an “input observation” is one or more numbers that are used as input to a machine learning model.

As used herein, the “health state” of the subject is the entire physical state of the subject’s body. The medical state may have various aspects including, for example, the subject’s height and weight, the subject’s blood pressure, temperature, and heart rate, the subject’s blood chemistry (e.g., the concentrations, in the blood, of glucose, cortisol, cholesterol, lactate, or ethanol, or a medication or therapy such as a statin), other characteristics of the blood (e.g., the prothrombin time, activated partial thromboplastin time, or thrombin time), and the presence of any medical conditions (e.g., (i) whether or not the subject has hypertension, cancer, dehydration or diabetes, or is suffering from a heart attack, kidney failure, sepsis or stroke, or (ii) the severity or likelihood of any such condition). The subject may be a human subject or an animal. In some embodiments, analogous methods as those disclosed herein for human or animal subjects may be employed to estimate the health state of another living organism, e.g., a plant, or to estimate the state of another object (e.g., the safety and quality of a food product in a factory for making such products).

As mentioned above, the estimate may consist of one or more numbers. For example, if the estimate is an estimate of the blood glucose level, it may be a single number (e.g., representing the blood glucose level, e.g., in units of mg/dL). As another example, if the estimate instead or also represents the presence of a heart attack, sepsis, or stroke, the presence of these three conditions may be represented as a vector of three numbers, the first of which, for example, may be (i) a number that is zero or one, depending on whether the machine learning model estimates that the subject is suffering from a heart attack, or (ii) a number between zero and one representing the estimated severity of a heart attack (with zero corresponding to the absence of a heart attack), or (iii) a number between zero and one representing the estimated likelihood that the subject is suffering from a heart attack (with, in this case also, zero corresponding to the absence of a heart attack). As used herein, a “medical condition” is an aspect of the medical state, and means a respect in which the medical state of the subject may differ from that of a reference subject, where the reference subject may be, for example, an average or typical subject, or a hypothetical perfectly healthy subject, or the state of the subject at a previous point in time. A medical condition may be, but need not be, disadvantageous; for example, an unusually high aerobic capacity is a medical condition as that term is used herein.

The training may be conducted, using a suitable cost function, at 110 (in FIG. 1), to cause the machine learning model to associate certain combinations of measurement values (which may be considered to be, or arranged as, a measurement vector or array) with certain aspects of the subject’s medical state. For example, the training data set may include, for each of the training subjects, a tissue spectrum as the input observation X, and a blood glucose level (separately measured, e.g., by chemical analysis of a blood sample) as the aspect, of the medical states of the training subjects, that may form the set of target variables Y. The cost function may be a function of the difference between (i) an estimate, of the blood glucose level, produced by the machine learning model and (ii) the corresponding target variable, e.g., the separately measured blood glucose level. After completing such training, the machine learning model may be capable of estimating the aspects of the medical state of the subject with which it was trained (e.g., the blood glucose level) from the measurement of a subject (e.g., the tissue spectrum of the subject). In cases where the target variable is taken from the health state of the subject at a later time than that of the input observations, the machine learning model may be capable of estimating aspects of the medical state of a subject at a later time, in other words, of predicting a future medical state. The training may result in the adjustment of parameters in the machine learning model so as to reduce or minimize the cost function. As used herein, “minimizing” the cost function means adjusting the parameters (e.g., iteratively) until a termination condition is satisfied. The termination condition may be satisfied, e.g., if the number of iterations that have been performed has reached a threshold, or if the improvement (reduction) in the cost function, per iteration, has fallen below a threshold. If the machine learning model is a neural network, the training may result in the adjustment of weights in the neural network). The set of values that these parameters take after training may be referred to as the “trained state” of the machine learning model. The parameter adjustment process can be carried out using stochastic gradient descent, in the case of a neural network model.

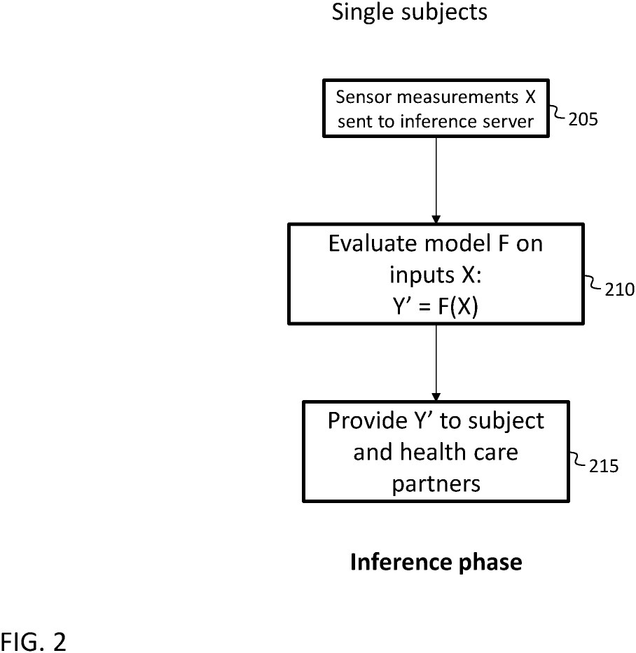

Once the machine learning model has been trained, the trained state of the machine learning model may be deployed to one or more inference servers, if these inference servers are separate from the central server (in some embodiments the central server 305 is the inference server or one of the inference servers). Equivalently, a plurality of inference servers, connected to each other or each connected to a central server may be considered to be a single distributed server. When performing an inference process, as illustrated in FIG. 2, the trained machine learning model may, at 205, receive an input observation (e.g., sensor measurements X), and generate, at 210, an estimate, e.g., estimates Y′ of aspects of the subject’s medical state, from the input observation. For example, the inference server may estimate the subject’s blood glucose level from a tissue spectrum of the subject. A portion of the estimate may then, at 215, be sent to the subject, or to the subject’s health care providers. In some embodiments, training may continue after the inference phase has begun, e.g., the parameters of the machine learning model may periodically or continuously be adjusted, to minimize the cost function, as new training data become available (e.g., from the training subjects, or from the subject (as discussed in further detail below)).

The parameters of the machine learning model may be, e.g., global parameters (based on “global” training data from all training subjects), or they may be subject-specific parameters (or some parameters may be global, and some may be subject-specific). The parameters may be stored in the inference server, or in a device (e.g., in a sensor, or in an interface device such as a mobile device with a display that is configured to communicate with a sensor and with the inference server) associated with a sensor or with a subject.

In some embodiments, subject-specific training data may be obtained. For example, if a subject obtains independent confirmation (or refutation) of an estimate (e.g., the presence of diabetes) generated by the machine learning model, the subject’s input observation (e.g., the subject’s tissue spectrum) and the subject’s target variable (e.g., the independently determined presence or absence of diabetes) may be used as subject-specific training data. The subject-specific data may be (i) added to the (global) training data set, or (ii) used to perform subject-specific training. Subject-specific training may involve, e.g., using a set of parameter values obtained by training the machine learning model with the global training data set as a set of initial values, and then adjusting some or all of the parameters (with, in some embodiments, some parameters being frozen) to minimize the cost function for (i) the global training data set, augmented with the subject-specific training data, or (ii) the subject-specific training data only.

The measurement or measurements upon which the estimate or estimate generated by the machine learning model are based may be high-dimensional data which may be, or include, measurements made by a wide range of methods for obtaining information about the subject, including, for example (as mentioned above), one or more tissue spectra (e.g., obtained at one or more locations on the subject’s body or obtained at one or more points in time), chemical analyses of samples (e.g., blood, saliva, urine or breath samples), physical measurements (e.g., height, weight, age, skin tone, temperature, strength, heart rate, blood pressure, or breathing rate), text (e.g. clinical notes, electronic medical records), or images (e.g., images of a portion of the subject’s skin, x-ray images, computer assisted tomography (CAT) scan images, magnetic resonance imaging (MRI) images, ultrasound images, or positron emission tomography (PET) scan images). In general, a measurement of a subject may be a measurement of any aspect (or any set of aspects) of the medical state of the subject, and any such measurements may be part of the input observation.

Moreover, such measurements, or data derived from them, may instead be part of the target variable used for training. For example, a tissue spectrum may be a portion of the input observation in a machine learning model used to estimate a blood glucose level from tissue spectra, as explained above. In other embodiments, a tissue spectrum may be used to infer an aspect of the subject’s medical state, and these derived data may be part of the target variable used for training (e.g., a blood alcohol level inferred from a tissue spectrum may be used to train a machine learning model to estimate blood alcohol level from other measurements of the subject). In some embodiments, the target variables used for training may include opinions of human experts (e.g., opinions regarding, the presence of a certain medical condition in a subject).

As mentioned above, a sensor may be a wearable spectrophotometer which (i) illuminates the skin of a subject with a probe beam including light of different wavelengths and (ii) measures light returning to the spectrophotometer, e.g., after scattering from subcutaneous tissue of the subject. For example, the spectrophotometer may produce a probe beam with light at, e.g., each of 100 different wavelengths in turn, and, for each wavelength, measure the intensity of the returning light. The wavelengths may be near infrared (NIR) wavelengths, which may be in the range from 780 nm to 2500 nm, or, e.g., from 1200 nm to 1800 nm. The ratio, as a function of wavelength, of (i) the intensity of the light returning to the spectrophotometer to (ii) the power of the probe beam, may depend on, and therefore provide information about, the subcutaneous tissue. Such a ratio, as a function of wavelength may (as mentioned above) be referred to as a “spectrum” or “tissue spectrum” of the subject. Such information may include, for example, information about the chemical composition of the subcutaneous tissue. As used herein, “tissue” refers to any biological component of the subject, including, for example, the skin of the subject, and blood in subcutaneous blood vessels of the subject. In some embodiments, a subject may be fitted with more than one such sensor; e.g., the subject may wear one sensor on the wrist and another on the chest.

The tissue spectrum may be represented in any of a number of ways. For example, a tissue spectrum may be represented as a vector, or array, of numbers, one for each wavelength of operation of the spectrophotometer, each number being the ratio at the respective wavelength, of (i) the intensity of the light returning to the spectrophotometer to (ii) the power of the probe beam. In other embodiments the tissue spectrum may be represented otherwise, e.g., using a representation that is independent of the wavelengths (and of the number or wavelengths) of operation of the spectrophotometer. For example, the tissue spectrum may be approximated by a continuous function, and converted to a spectrophotometer-independent representation by resampling the continuous function at another set of wavelengths. As another example, the continuous function may be defined by a set of parameters (e.g., polynomial coefficients, if the continuous function is, e.g., a polynomial or a cubic spline, or a set of peak magnitudes and peak widths if the continuous function is or includes a superposition of one or more peaks, each peak being a Lambertian function), and the set of parameters may be employed as a spectrophotometer-independent representation of the tissue spectrum. The use of a spectrophotometer-independent representation may be of use, for example, if different spectrophotometers are used by different subjects or at different times.

As mentioned above, the input observation may include a time series of similarly-obtained measurements, e.g., a series of tissue spectra. Similarly, a time series of other measurements may be, or be part of, the input observation used for performing an inference process. For example, the input observation may include a time series of heart rate measurements, a time series of blood test results, or a series of MRI images. If a time series of tissue spectra is used, the spectra may be obtained at a rate, for example, of 10 or 20 Hz, so that variations with the cardiac cycle may be resolvable in the data. Such a time series of tissue spectra may form a two-dimensional array, with one dimension corresponding to time and the other dimension corresponding to wavelength.

Various types of machine learning models may be used, including neural networks (e.g., convolutional neural networks), support vector machines, partial least squares algorithms, and decision trees. If the input observation is or includes a time series of measurements, a transformer or a recurrent neural network may be used as the machine learning model. For example, in the case of a spectrophotometer sensor with 194 wavelengths, a 5-layered 1-dimensional convolutional neural network may be used to estimate the level of a particular blood analyte (e.g., to estimate the level of a chemical component of the blood). The input layer may have 2 feature maps that are 9x1, then 2 feature maps that are 7x1, then 4 feature maps that are 7x1, then 8 feature maps that are then 5x1, then 12 feature maps that are 3x1, followed by a fully-connected layer with a single output, where each convolution operation is followed by a rectified linear operation, then a batch normalization step, then an average pooling layer.

As an alternative, a partial least squares (PLS) model may be used as the machine learning model. In the case of a spectrophotometer sensor with 194 wavelengths, the matrix X can be constructed by arranging n rows of 194-dimensional observations into a n by 194 matrix of predictors, and the matrix Y can be constructed by arranging n rows of p dimensional target variables, then applying the PLS1 algorithm to estimate factor and loading matrices and error matrices given X and Y. As an alternative, a gradient-boosted decision tree model may be used as the machine learning model. In the case of a spectrophotometer sensor with 194 wavelengths, X and Y matrices may be built as in the PLS case, and an ensemble of 10 decision trees with a maximum depth of 5 may be built using the XGBoost algorithm with either a linear or logistic objective function for regression or classification problems, respectively. As an alternative, a regularized linear regression model, such as LASSO or ridge regression, may be used as a machine learning model. Using the example above, one may set X as the matrix of independent variables and Y as the matrix of dependent variables and apply either the LASSO algorithm with a L1 maximum norm parameter or the ridge regression algorithm with a biasing constant k of 0.001.

As an alternative, a machine learning model may be used that carries out unsupervised learning. In unsupervised learning, the machine learning algorithm uses only the X matrix as input. One example of unsupervised learning is clustering, where the algorithm uses only the X matrix and outputs an estimated “class” for each observation (in this example, each row) of the X matrix. For example, one may use a clustering algorithm, such as the k-means algorithm with a value of k=2, on the X matrix to divide observations into two classes, which could be used as an estimate of whether an observation came from a diabetic on non-diabetic subject. Another type of unsupervised learning is dimensionality reduction, which takes the observation matrix X and solves for a transformation T that maps each observation into a lower dimensional space while preserving specified properties of the data. For example, one may use principal components analysis (PCA) on the X matrix to solve for a set of basis vectors such that the projections of the data onto these vectors are uncorrelated in the dataset. Moreover, by retaining only the projections corresponding to the top N eigenvalues (where N may be 10, for example), one may project the data to an N-dimensional subspace that can be used as input to a downstream machine learning model.

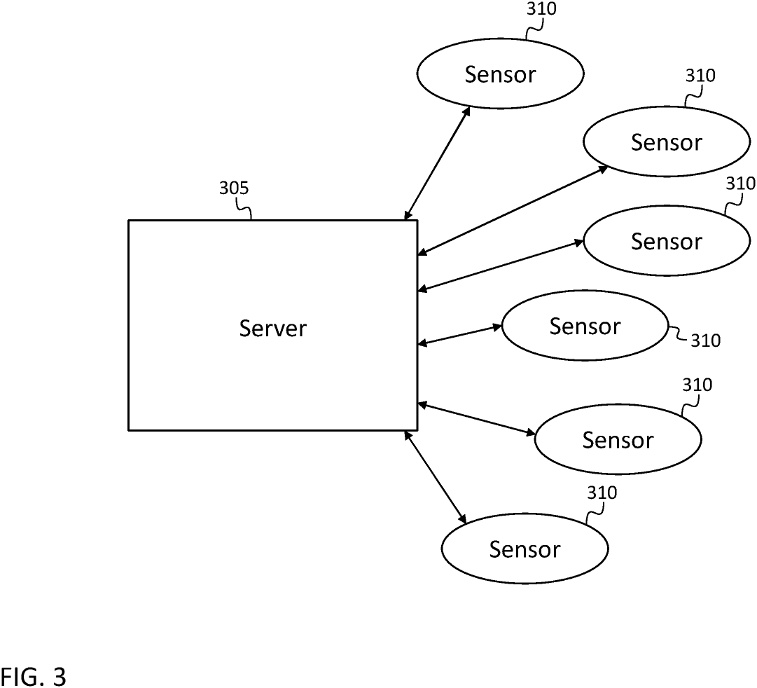

Referring to FIG. 3, in some embodiments, a central server 310 is connected to a plurality of sensors 310 (e.g., portable or wearable spectrophotometers, as discussed above), each of which takes, from time to time, or substantially continuously (e.g., with a sample rate of 10 Hz or 20 Hz), measurements of a respective subject (or, in some cases, several of which may be taking measurements of the same subject). During training of the machine learning model, the sensors 310 relay the measurements to the central server 305. During training, target variable data are also sent to the central server 305. The target variable data may be sent separately, or over shared data paths. For example, each sensor may be a wearable spectrophotometer worn by a respective subject. Each subject may also carry a portable device such as a mobile phone, which may (i) receive the measurements via a local (e.g., WiFi or Bluetooth) data connection and relay the measurements to the central server 305 through the Internet (e.g., via a mobile telephone network). The subject may also obtain target variable data (e.g., using an additional sensor) and transmit the data to the central server 305 through the same connection between the mobile device and the central server 305. For example, if the machine learning model is being trained to estimate blood glucose levels, the subject may have a blood glucose measuring device that displays blood glucose level measurements to the subject, who then keys these measurements into the mobile device (or the blood glucose measuring device may communicate these measurements directly with the mobile device through a local connection) for transmission to the central server 305.

The inference process may also employ the configuration of FIG. 3. During inference, an interference server (which may be the same central server 305, or a separate server) may similarly receive sensor data from a plurality of sensors. In this case the inference server may generate estimates of aspects of the subject’s medical state. These estimates may be, for example, (i) sent back to the subject, (ii) sent to the subject’s healthcare provider, or (iii) compared to prescribed ranges, and, if they fall within such ranges, used to trigger additional steps, such as alerts (e.g., a warning to the subject if an estimated blood glucose level is dangerously high), medication, or other interventions.

In some situations, for example, if the available data rate of the connection to the central server 305 is limited, or if sending data to the central server 305 is costly, a portion of the data generated by the sensor may be replaced with a lower volume of processed data. For example, outliers, or data that are of relatively low value for inference (and for training) may be dropped, or averaged with other data. For example, if blood glucose level is to be estimated, wavelengths at which the spectrum varies relatively little as a function of blood glucose level may be omitted from the input observation. As another example, if the raw data obtained by the sensor vary with the cardiac cycle, then the raw data may be fit to the cardiac cycle, and lower-rate average data may be transmitted to the central server 305.

As used herein, the “presence of” a substance or a medical condition means whether or not the substance or medical condition is present, or the extent to which it is present (e.g., the severity of a medical condition or the concentration of a substance in the blood), or the likelihood of the substance or medical condition being present. As such, “estimating the presence of” something means estimating whether or not the thing is present, or estimating the extent to which the thing is present, or estimating the likelihood of the thing being present.

As used herein, “a portion of” something means “at least some of” the thing, and as such may mean less than all of, or all of, the thing. As such, “a portion of” a thing includes the entire thing as a special case, i.e., the entire thing is an example of a portion of the thing. As used herein, the word “or” is inclusive, so that, for example, “A or B” means any one of (i) A, (ii) B, and (iii) A and B. As used herein, the term “array” refers to an ordered set of numbers regardless of how stored (e.g., whether stored in consecutive memory locations, in a linked list, or in another manner).

The central server and each of the one or more inference servers may be, or include a processing circuit configured to perform portions of the methods disclosed herein. The term “processing circuit” is used herein to mean any combination of hardware, firmware, and software, employed to process data or digital signals. Processing circuit hardware may include, for example, application specific integrated circuits (ASICs), general purpose or special purpose central processing units (CPUs), digital signal processors (DSPs), graphics processing units (GPUs), and programmable logic devices such as field programmable gate arrays (FPGAs). In a processing circuit, as used herein, each function is performed either by hardware configured, i.e., hard-wired, to perform that function, or by more general-purpose hardware, such as a CPU, configured to execute instructions stored in a non-transitory storage medium. A processing circuit may be fabricated on a single printed circuit board (PCB) or distributed over several interconnected PCBs. A processing circuit may contain other processing circuits; for example, a processing circuit may include two processing circuits, an FPGA and a CPU, interconnected on a PCB.

As used herein, when a method (e.g., an adjustment) or a first quantity (e.g., a first variable) is referred to as being “based on” a second quantity (e.g., a second variable) it means that the second quantity is an input to the method or influences the first quantity, e.g., the second quantity may be an input (e.g., the only input, or one of several inputs) to a function that calculates the first quantity, or the first quantity may be equal to the second quantity, or the first quantity may be the same as (e.g., stored at the same location or locations in memory as) the second quantity.

Although exemplary embodiments of a system and method for monitoring indicators of patient health have been specifically described and illustrated herein, many modifications and variations will be apparent to those skilled in the art. Accordingly, it is to be understood that a system and method for monitoring indicators of patient health constructed according to principles of this disclosure may be embodied other than as specifically described herein. The invention is also defined in the following claims, and equivalents thereof.

1. A method, comprising:
receiving a first measurement of a subject;
receiving a second measurement of the subject, obtained after the first measurement; and
generating, using a machine learning inference process based on the first measurement and on the second measurement, an estimate of an aspect of the health state of the subject.

2. The method of claim 1, wherein the aspect of the health state of the subject is a concentration of a chemical constituent of a tissue of the subject.

3. The method of claim 2, wherein the chemical constituent is a substance selected from the group consisting of glucose, cortisol, cholesterol, lactate, ethanol, and water.

4. (canceled)

5. The method of claim 1, wherein:
the first measurement is a first tissue spectrum of the subject, and
the second measurement is a second tissue spectrum of the subject.

6. The method of claim 5, further comprising receiving a third measurement of the subject, wherein the machine learning inference process is further based on the third measurement.

7. The method of claim 6, wherein:
the third measurement is a third tissue spectrum of the subject, and
the first tissue spectrum and the third tissue spectrum are obtained at different locations on the body of the subject.

8. (canceled)

9. (canceled)

10. The method of claim 1, further comprising performing a machine learning training process to generate a trained state, wherein
the machine learning training process is based on a plurality of measurements of one or more training subjects; and
the machine learning inference process is further based on the trained state.

11. (canceled)

12. The method of claim 10, wherein the machine learning training process comprises clustering.

13. The method of claim 10, wherein the machine learning training process comprises dimensionality reduction.

14. (canceled)

15. (canceled)

16. A method, comprising:
receiving a first measurement of a subject, the first measurement being a first tissue spectrum of the subject; and
generating, using a machine learning inference process based on the first measurement, an estimate of an aspect of the health state of the subject.

17-20. (canceled)

21. The method of claim 16, further comprising receiving a second measurement of the subject, wherein the machine learning inference process is further based on the second measurement.

22. The method of claim 21, wherein the second measurement is a second tissue spectrum of the subject.

23. The method of claim 22, wherein the first tissue spectrum and the second tissue spectrum are obtained at different points in time.

24. The method of claim 22, wherein the first tissue spectrum and the second tissue spectrum are obtained at different locations on the body of the subject.

25. The method of claim 21, wherein the second measurement of the subject is a result of a chemical analysis of a sample from the subject.

26. The method of claim 21, wherein the second measurement of the subject is an image of a portion of the subject.

27-31. (canceled)

32. A system, comprising:
a processing circuit, the processing circuit being configured to:
receive a first measurement of a subject, the first measurement being a first tissue spectrum of the subject; and
generate, using a machine learning inference process based on the first measurement, an estimate of an aspect of the health state of the subject.

33. The system of claim 32, wherein the aspect of the health state of the subject is a concentration of a chemical constituent of a tissue of the subject.

34. (canceled)

35. The system of claim 32, wherein:
the processing circuit is further configured to receive a second measurement of a subject, the second measurement being a second tissue spectrum of the subject, obtained later than the first measurement; and
the generating of the estimate of the aspect of the health state of the subject comprises using a machine learning inference process further based on the second measurement.

36. The system of claim 32, further comprising a portable spectrophotometer, configured to obtain a plurality of tissue spectra including the first tissue spectrum.

37. (canceled)
